(19) United States
(12) Patent Application Publication
(10) Pub. No.: US 2008/0039695 A1
(43) Pub. Date: Feb. 14, 2008

(54) Fluorescence endoscope system, fluoroscopy apparatus, fluoroscopy method, fluorescence-information processing apparatus, and fluorescence-information processing method

(75) Inventors: Hideyuki Takaoka, Tokyo (JP); Akira Hasegawa, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/890,964

(22) Filed: Aug. 8, 2007

(30) Foreign Application Priority Data
Aug. 14, 2006 (JP) 2006-221107

(51) Int. Cl. A61B 1/06

(52) U.S. Cl. 600/178

(57) ABSTRACT

The invention provides a fluorescence endoscope system, a fluoroscopy apparatus, a fluoroscopy method, a fluorescence-information processing apparatus, and a fluorescence-information processing method which allow superior fluoroscopy by reducing the effect of autofluorescence. Provided are an excitation-light radiating part for irradiating an object under examination in a body cavity with excitation light having a wavelength included in an absorption spectrum of a fluorescent dye deposited on or absorbed in the object under examination in a state where the fluorescent dye is not deposited on or absorbed in the object under examination; a correction-information acquiring part for acquiring correction information that can be used to correct for the effect of autofluorescence of the object under examination on observation results of fluorescence emitted from the fluorescent dye deposited on or absorbed in the object under examination upon irradiation with the excitation light, based on observation results of the object under examination upon irradiation with the excitation light by the excitation-light radiating part.

### BACKGROUND OF THE INVENTION

1. Field of the Invention

The present invention relates to fluorescence endoscope systems for fluoroscopy of an object under examination, fluoroscopy apparatuses, fluoroscopy methods, fluorescence-information processing apparatuses, and fluorescence-information processing methods.

This application is based on Japanese Patent Application No. 2006-221107, the content of which is incorporated herein by reference.

2. Description of Related Art

In endoscopy, the observation of fluorescence images is useful in diagnosis and examination of diseases because the fluorescence images provide different biological information from that provided by reflection images.

For example, the concentration distribution of a disease-derived substance can be observed on a fluorescence image acquired by applying a fluorescent probe that changes from being nonfluorescent to fluorescent in response to the substance.

In addition, changes in a living organism and the appearance of a disease can be observed using autofluorescence, that is, fluorescence emitted from the living organism itself.

Reflection images, on the other hand, contain useful information that is different from that contained in fluorescence images.

Specifically, a reflection image can be used to observe, for example, the density and appearance of blood vessels, thus providing information about diseases, such as inflammation.

One known fluorescent probe that emits fluorescence when bound to a disease-derived substance is a fluorescent dye capable of in vivo visualization of an affected area, such as tumor/cancer tissue, with high sensitivity. Some methods using 5-aminolevulinic acid, an exogenous substance, have already been put to clinical use as methods for cancer diagnosis (for example, see Kennedy, J. C. et al., J. Photochem. Photobiol. B 6:143 1990).

In fluoroscopy using fluorescent probes, however, irradiation of biological tissue under examination with excitation light has the side effect of inducing autofluorescence from the biological tissue. This autofluorescence causes noise in fluoroscopy using fluorescent probes. Accordingly, there are demands for improved fluoroscopy in which the influence of autofluorescence is reduced.

### BRIEF SUMMARY OF THE INVENTION

The present invention provides the following solutions.

The first aspect of the present invention provides a fluorescence endoscope system for observing fluorescence emitted from a fluorescent dye deposited on or absorbed in biological tissue in a body cavity of a living organism by inserting at least part of the system into the body cavity and irradiating the biological tissue with excitation light. The system includes an excitation-light radiating part configured to irradiate the biological tissue with excitation light having a wavelength included in an absorption spectrum of the fluorescent dye in a state where the fluorescent dye is not deposited on or absorbed in the biological tissue; and a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

In the fluoroscopy of the biological tissue using the above fluorescence endoscope system, the biological tissue is observed while being irradiated with the excitation light from the excitation-light radiating part in the state where the fluorescent dye is not deposited on or absorbed in the biological tissue (this observation is hereinafter referred to as “observation for correction-information acquisition”) before or after the fluorescence observation of the biological tissue using the fluorescent dye.

The wavelength of the excitation light used for the observation for correction-information acquisition lies in the absorption spectrum of the fluorescent dye. In the observation, therefore, the biological tissue emits autofluorescence similar to the autofluorescence emitted in the fluorescence observation using the fluorescent dye.

According to the observation results acquired by the observation for correction-information acquisition, the correction-information acquiring part acquires the “correction information that can be used to correct for the effect of autofluorescence of the biological tissue” on the results of the fluorescence observation using the fluorescent dye (for example, information about the intensity and intensity distribution of light, other than the fluorescence of the fluorescent dye, emitted from the biological tissue upon irradiation with the excitation light for exciting the fluorescent dye).

The state where the fluorescent dye is not deposited on or absorbed in the biological tissue may be a state before the fluorescent dye is deposited on or absorbed in the biological tissue.

The correction-information acquiring part may also function as a fluorescence-information acquiring part configured to acquire information about the fluorescence emitted from the fluorescent dye deposited on or absorbed in the biological tissue.

The correction-information acquiring part may also function as an image-acquisition part configured to acquire information about the fluorescence emitted from the fluorescent dye deposited on or absorbed in the biological tissue as image information.

The fluorescence endoscope system may further include a correction-information storing part configured to store the correction information acquired by the correction-information acquiring part.

The fluorescence endoscope system may further include a correcting part configured to correct for the effect of autofluorescence of the biological tissue on the observation results of the fluorescence emitted from the fluorescent dye deposited on or absorbed in the biological tissue, based on the correction information acquired by the correction-information acquiring part.

The fluorescent dye may have the ability to selectively stain normal cells and tumor cells in the biological tissue.

The second aspect of the present invention further provides a fluoroscopy apparatus for observing fluorescence emitted from a fluorescent dye deposited on or absorbed in biological tissue in a body cavity of a living organism by irradiating the biological tissue with excitation light. The apparatus includes an excitation-light radiating part configured to irradiate the biological tissue with excitation light having a wavelength included in an absorption spectrum of the fluorescent dye in a state where the fluorescent dye is not deposited on or absorbed in the biological tissue; and a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

In the fluoroscopy of the biological tissue using the above fluoroscopy apparatus, the observation for correction-information acquisition is performed before or after the fluorescence observation of the biological tissue using the fluorescent dye.

Thus, the correction-information acquiring part acquires the correction information that can be used to correct for the effect of autofluorescence of the biological tissue on the results of the fluorescence observation using the fluorescent dye.

The third aspect of the present invention further provides a fluoroscopy apparatus for observing fluorescence emitted from a fluorescent dye deposited on or absorbed in biological tissue extracted and removed from a living organism by irradiating the biological tissue with excitation light. The apparatus includes an excitation-light radiating part configured to irradiate the biological tissue with excitation light having a wavelength included in an absorption spectrum of the fluorescent dye in a state where the fluorescent dye is not deposited on or absorbed in the biological tissue; and a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

In the fluoroscopy of the biological tissue using the above fluoroscopy apparatus, the observation for correction-information acquisition is performed on the biological tissue extracted and removed from the living organism before or after the fluorescence observation of the biological tissue using the fluorescent dye.

Thus, the correction-information acquiring part acquires the correction information that can be used to correct for the effect of autofluorescence of the biological tissue on the results of the fluorescence observation using the fluorescent dye.

The fourth aspect of the present invention further provides a fluoroscopy method for observing fluorescence emitted from a fluorescent dye deposited on or absorbed in biological tissue extracted and removed from a living organism by irradiating the biological tissue with excitation light. The method includes an observation step, used for correction-information acquisition, in which the biological tissue is irradiated with excitation light having a wavelength included in an absorption spectrum of the fluorescent dye in a state where the fluorescent dye is not deposited on or absorbed in the biological tissue and observation results of the biological tissue upon irradiation with the excitation light are acquired; and a correction-information acquiring step of acquiring correction information that can be used to correct for the effect of autofluorescence of the biological tissue on observation results of the fluorescence emitted from the fluorescent dye deposited on or absorbed in the biological tissue upon irradiation with the excitation light, based on the observation results, acquired in the observation step used for correction-information acquisition, of the biological tissue upon irradiation with the excitation light.

In the above fluoroscopy method, the observation for correction-information acquisition is performed on the biological tissue extracted and removed from the living organism before or after the fluorescence observation of the biological tissue using the fluorescent dye (observation step used for correction-information acquisition). The observation results are used to acquire the correction information that can be used to correct for the effect of autofluorescence of the biological tissue on the results of the fluorescence observation using the fluorescent dye (correction-information acquiring step).

The fifth aspect of the present invention further provides a fluorescence-information processing apparatus for processing information about fluorescence emitted from a fluorescent dye deposited on or absorbed in biological tissue by irradiating the biological tissue with excitation light. The apparatus includes an correction-information storing part configured to store information about observation results of the biological tissue upon irradiation with excitation light having a wavelength included in an absorption spectrum of the fluorescent dye in a state where the fluorescent dye is not deposited on or absorbed in the biological tissue; and a correcting part configured to correct for the effect of autofluorescence of the biological tissue on observation results of the fluorescence emitted from the fluorescent dye deposited on or absorbed in the biological tissue upon irradiation with the excitation light, based on the information stored in the correction-information storing part.

In the above fluorescence-information processing apparatus, the observation for correction-information acquisition is performed before or after the fluorescence observation of the biological tissue using the fluorescent dye, thus acquiring the correction information that can be used to correct for the effect of autofluorescence of the biological tissue on the results of the fluorescence observation using the fluorescent dye.

The sixth aspect of the present invention further provides a fluorescence-information processing method for processing information about fluorescence emitted from a fluorescent dye deposited on or absorbed in biological tissue by irradiating the biological tissue with excitation light. The method includes a correction-information acquiring step of acquiring information about observation results of the biological tissue upon irradiation with excitation light having a wavelength included in an absorption spectrum of the fluorescent dye in a state where the fluorescent dye is not deposited on or absorbed in the biological tissue; and a correction step of correcting for the effect of autofluorescence of the biological tissue on observation results of the fluorescence emitted from the fluorescent dye deposited on or absorbed in the biological tissue upon irradiation with the excitation light, based on the information about the observation results acquired in the correction-information acquiring step.

In the above fluorescence-information processing method, the observation for correction-information acquisition is performed before or after the fluorescence observation of the biological tissue using the fluorescent dye, thus acquiring the correction information that can be used to correct for the effect of autofluorescence of the biological tissue on the results of the fluorescence observation using the fluorescent dye (correction-information acquiring step).

Accordingly, the fluorescence endoscope system, the fluoroscopy apparatus, the fluoroscopy method, the fluorescence-information processing apparatus, and the fluorescence-information processing method according to the present invention allow superior fluoroscopy by reducing the effect of autofluorescence of biological tissue by using correction information that can be used to correct for the effect of autofluorescence.

### BRIEF DESCRIPTION OF THE SEVERAL VIEWS OF THE DRAWINGS

FIG. 1 is a block diagram of the overall structure of a fluorescence endoscope system according to a first embodiment of the present invention;

FIG. 2 is a schematic diagram of the interior of an image-acquisition unit of the fluorescence endoscope system shown in FIG. 1;

FIG. 3 is a set of graphs showing the transmittance characteristics of optical components of the fluorescence endoscope system shown in FIG. 1 and the wavelength characteristics of irradiation light and fluorescence;

FIG. 4 is a timing chart illustrating the operation of the fluorescence endoscope system shown in FIG. 1;

FIG. 5 is a timing chart illustrating the operational modes of a valve control circuit of the fluorescence endoscope system shown in FIG. 1;

FIG. 6 is a timing chart illustrating an example of the switching of photometry modes during image acquisition;

FIG. 7 is a block diagram of the overall structure of a fluorescence endoscope system according to a second embodiment of the present invention;

FIG. 8 is a set of graphs showing the transmittance characteristics of optical components of the fluorescence endoscope system shown in FIG. 7 and the wavelength characteristics of irradiation light and fluorescence;

FIG. 9 is a timing chart illustrating the operational modes of a valve control circuit of the fluorescence endoscope system shown in FIG. 7; and

FIG. 10 is a set of graphs showing the transmittance characteristics of the optical components of the fluorescence endoscope system shown in FIG. 7 and the wavelength characteristics of irradiation light and fluorescence in the case where a cyanine-based fluorescent dye/probe is used.

### DETAILED DESCRIPTION OF THE INVENTION

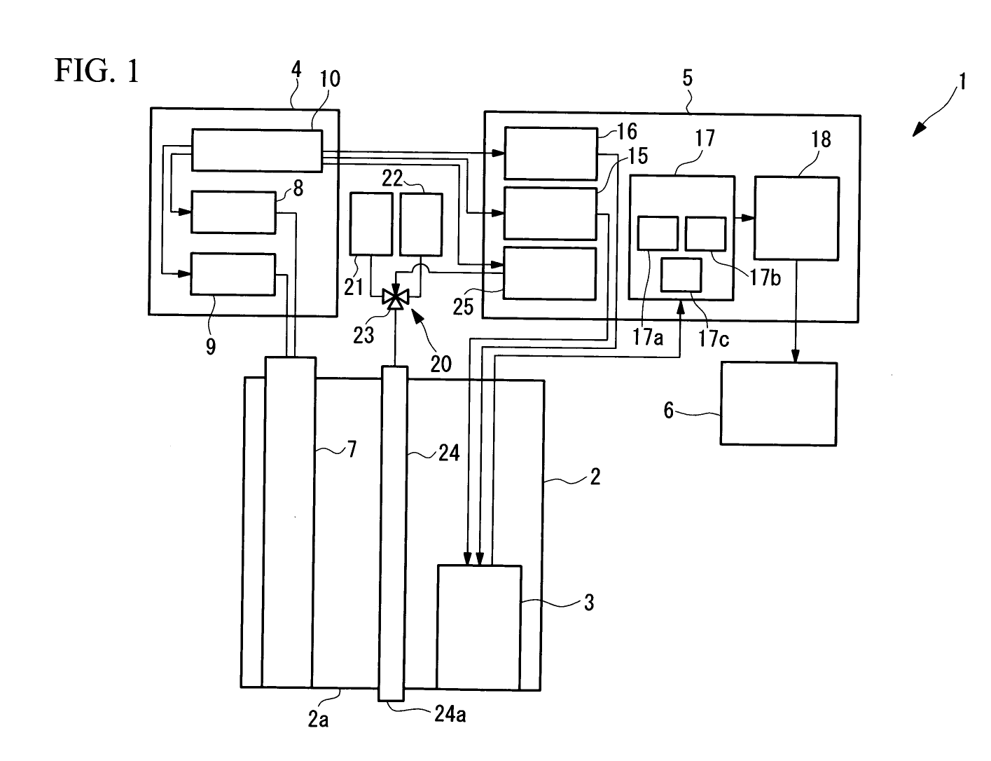

A fluorescence endoscope system 1 according to a first embodiment of the present invention will now be described with reference to FIGS. 1 to 5.

In FIG. 1, the fluorescence endoscope system 1 according to this embodiment includes an insertion part 2 for insertion into a body cavity of a living organism, an image-acquisition unit (image-acquisition part) 3 disposed in the insertion part 2, a light source unit (light source part) 4 for emitting different types of light, a feed unit 20 for supplying a liquid to be discharged from a front end 2a of the insertion part 2, a control unit (control part) 5 for controlling the image-acquisition unit 3, the light source unit 4, and the feed unit 20, and a display unit (output part) 6 for displaying an image acquired by the image-acquisition unit 3.

The insertion part 2 has outer dimensions small enough that it can be inserted into the body cavity of the living organism. The insertion part 2 accommodates the image-acquisition unit 3 and a light guide (light-guiding optical system) 7 through which light emitted from the light source unit 4 propagates to the front end 2a.

The light source unit 4 includes an illumination light source 8, an excitation light source 9, and a light-source control circuit 10. The illumination light source 8 illuminates an object under examination (biological tissue) in the body cavity with illumination light (irradiation light) to obtain light reflected by the object under examination. The excitation light source 9 irradiates the object under examination in the body cavity with excitation light to excite a fluorescent substance contained in the object under examination so that it emits fluorescence. The light-source control circuit 10 controls the light sources 8 and 9. The light source unit 4 and the light guide 7 thus constitute an excitation-light radiating part for irradiating the object under examination with excitation light.

The illumination light source 8 is, for example, a combination of a xenon lamp and a bandpass filter (not shown). The bandpass filter has a 50% transmission range of 420 to 450 nm. That is, the illumination light source 8 emits illumination light in the wavelength range of 420 to 450 nm.

The excitation light source 9 is, for example, a semiconductor laser that emits excitation light with a peak wavelength of 490±5 nm (or an argon laser that emits excitation light with a peak wavelength of 488±5 nm). With this wavelength, the excitation light can excite fluorescein-based esterase-sensitive fluorescent probes (fluorescent dyes). Fluoroscopy of biological tissue by the endoscope system 1 uses tumor cell-selective or tumor tissue-selective fluorescent dyes, including the above esterase-sensitive fluorescent probes.

For example, the fluorescent dye used in this embodiment can be, but is not limited to, a fluorescent probe disclosed in U.S. Pat. No. 6,696,241, as exemplified by a fluorescein ester selected from a group including fluorescein diacetate (FDA).

The light-source control circuit 10 alternately switches on and off the illumination light source 8 and the excitation light source 9 at a predetermined timing according to a timing chart described later.

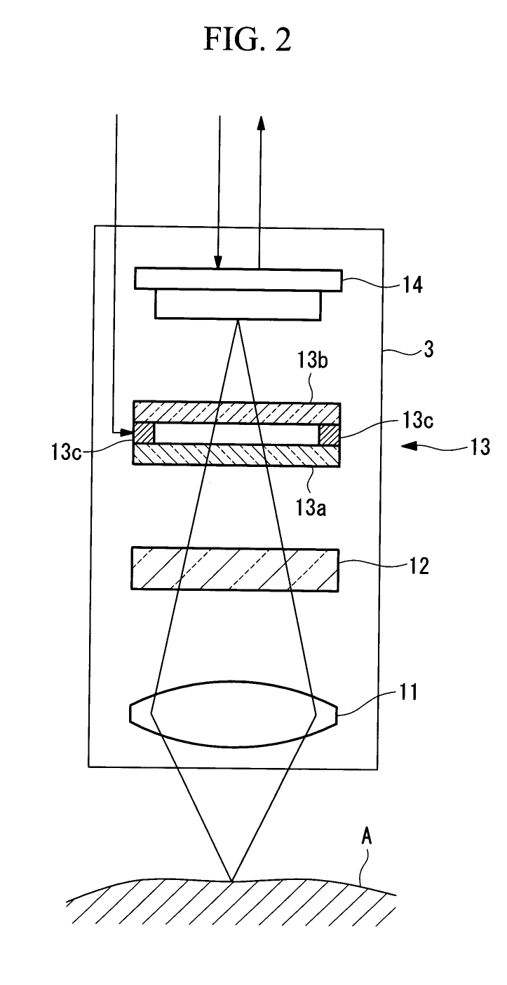

Referring to FIG. 2, the image-acquisition unit 3 includes an image-acquisition optical system 11 for collecting incident light from an object A under examination, an excitation-light cutting filter 12 for blocking excitation light from the object A under examination, a tunable-spectrum device (tunable-spectrum part) 13 whose spectral properties can be changed under the control of the control unit 5, and an image-acquisition device 14 for acquiring an image of the light collected by the image-acquisition optical system 11 and converting it into electrical signals.

The tunable-spectrum device 13 is an etalon-type optical filter including two parallel planar optical members 13a and 13b separated from each other and having reflective films laminated on opposing surfaces thereof and an actuator 13c for changing the distance between the two optical members 13a and 13b. The actuator 13c is, for example, a piezoelectric device. The actuator 13c of the tunable-spectrum device 13 can be operated to change the distance between the optical members 13a and 13b, thereby changing the wavelength range of transmission light.

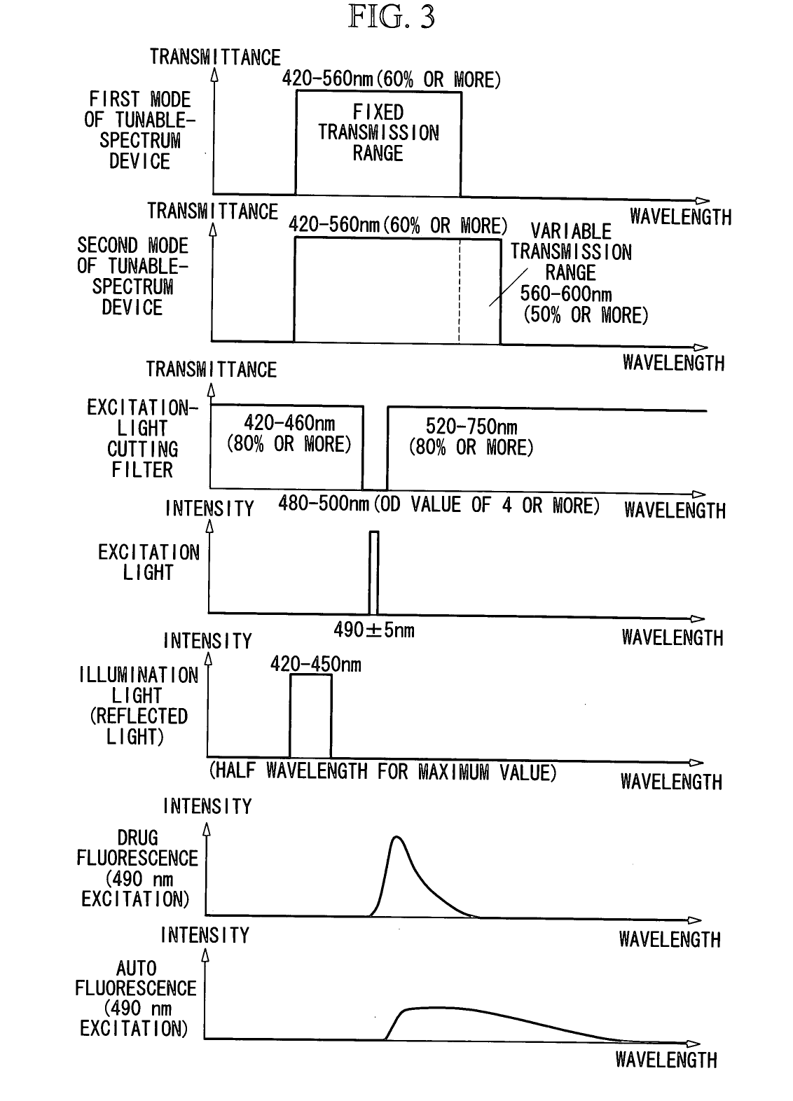

More specifically, the tunable-spectrum device 13 has a transmittance-wavelength characteristic with two transmission ranges, including one fixed transmission range and one variable transmission range, as shown in FIG. 3. In the fixed transmission range, the tunable-spectrum device 13 always transmits incident light irrespective of the mode of the tunable-spectrum device 13. In the variable transmission range, the transmittance characteristics vary with the mode of the tunable-spectrum device 13.

In this embodiment, the variable transmission range of the tunable-spectrum device 13 lies in the red wavelength range (for example, 560 to 600 nm). The tunable-spectrum device 13 changes between two modes in response to control signals transmitted from the control unit 5.

A first mode is a mode where the transmittance in the variable transmission range is sufficiently decreased in comparison with a second mode, so that the tunable-spectrum device 13 transmits drug fluorescence. The second mode is a mode where the transmittance in the variable transmission range is increased to 50% or more, so that the tunable-spectrum device 13 transmits reflected light of illumination light. In the first mode, as shown in FIG. 3, the tunable-spectrum device 13 blocks biological autofluorescence in the variable transmission range by sufficiently decreasing the transmittance in that range in comparison the second mode. Such autofluorescence would generate noise in the observation of drug fluorescence. The first mode thus allows transmission of drug fluorescence mainly in the fixed transmission range. The second mode, on the other hand, allows transmission of blue light, green light, and red light, which are necessary for white-light observation, with the fixed transmission range set to the range of, for example, 420 to 560 nm and the variable transmission range set to the range of, for example, 560 to 600 nm, as shown in FIG. 3.

The illumination light has a wavelength of, for example, 420 to 450 nm, as shown in FIG. 3. In this wavelength range, the light carries information about blood vessels. Alternatively, the illumination light can be red light (580 to 590 nm). In this wavelength range, less light is absorbed by the living organism, and the light carries the surface profile more clearly than blue light.

The fixed transmission range is set to the range of, for example, 420 to 560 nm, with a transmittance of 60% or more.

The fixed transmission range lies in a wavelength range including the wavelength of the reflected light of the illumination light, so that the reflected light can be transmitted to the image-acquisition device 14 both in the first mode and in the second mode.

The excitation-light cutting filter 12 has a transmittance of 80% or more in the wavelength range of 420 to 470 nm, an OD of 4 or more (=a transmittance of 1×10-4 or less) in the wavelength range of 480 to 500 nm, and a transmittance of 80% or more in the wavelength range of 520 to 750 nm.

Referring back to FIG. 1, the control unit 5 includes an image-acquisition-device drive circuit 15 for drive control of the image-acquisition device 14, a tunable-spectrum-device control circuit 16 for drive control of the tunable-spectrum device 13, a valve control circuit 25, a frame memory 17 for storing image information acquired by the image-acquisition device 14, and an image-processing circuit 18 for processing the image information stored in the frame memory 17 and supplying it to the display unit 6.

The image-acquisition-device drive circuit 15 and the tunable-spectrum-device control circuit 16 are connected to the light-source control circuit 10 so that they can execute the drive control of the image-acquisition device 14 and the tunable-spectrum device 13 in synchronization with the switching of the illumination light source 8 and the excitation light source 9 by the light-source control circuit 10.

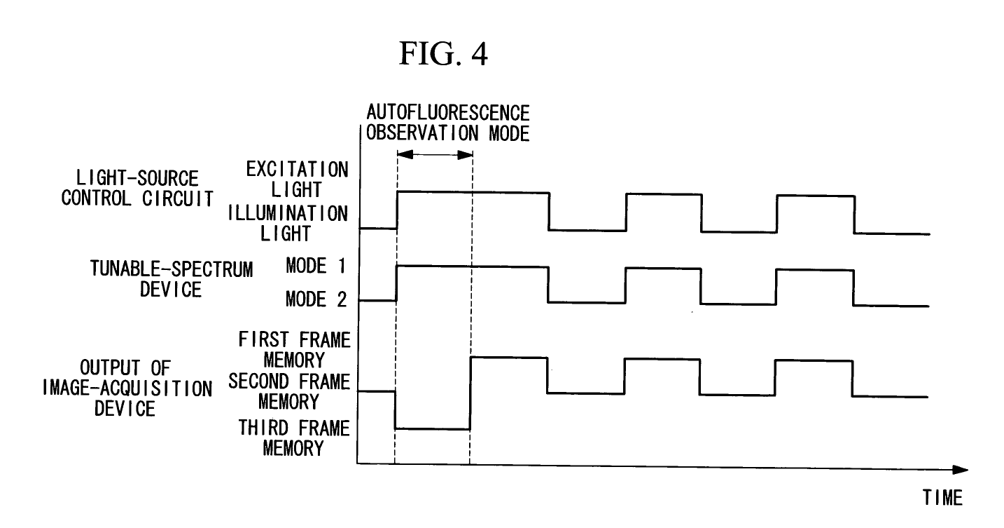

Referring to the timing chart of FIG. 4, when the light-source control circuit 10 drives the excitation light source 9 to emit excitation light, the tunable-spectrum-device control circuit 16 sets the tunable-spectrum device 13 to the first mode, and the image-acquisition-device drive circuit 15 supplies image information output from the image-acquisition device 14 to a first frame memory 17a. Thus, the image-acquisition unit 3, the image-acquisition-device drive circuit 15, and the tunable-spectrum-device control circuit 16 constitute a fluorescence-information acquiring part for acquiring information about fluorescence emitted from the fluorescent dye deposited on or absorbed in the object A under examination upon irradiation with excitation light. When the illumination light source 8 emits illumination light, the tunable-spectrum-device control circuit 16 sets the tunable-spectrum device 13 to the second mode, and the image-acquisition-device drive circuit 15 supplies image information output from the image-acquisition device 14 to a second frame memory 17b.

In this embodiment, the fluorescence endoscope system 1 has an operational mode termed autofluorescence observation mode.

In the autofluorescence observation mode, as shown in the timing chart of FIG. 4, the light-source control circuit 10 drives the excitation light source 9 to emit excitation light while the tunable-spectrum-device control circuit 16 sets the tunable-spectrum device 13 to the first mode and the image-acquisition-device drive circuit 15 supplies image information (autofluorescence information) output from the image-acquisition device 14 to a third frame memory 17c. Thus, the image-acquisition device 14, the image-acquisition-device drive circuit 15, the tunable-spectrum-device control circuit 16, and the third frame memory 17c constitute a correction-information acquiring part for acquiring correction information (autofluorescence information) that can be used to correct for the effect of autofluorescence on the observation results of fluorescence emitted from the fluorescent dye deposited on or absorbed in the object A under examination upon irradiation with excitation light. The third frame memory 17c constitutes a correction-information storing part for storing the correction information.

The image-processing circuit 18, for example, receives fluorescence image information acquired by irradiation with excitation light from the first frame memory 17a and supplies it to a red channel of the display unit 6, and also receives reflected-light image information acquired by irradiation with illumination light from the second frame memory 17b and supplies it to a green channel of the display unit 6. In addition, for example, the image-processing circuit 18 receives fluorescence image information acquired in autofluorescence observation by irradiation with excitation light from the third frame memory 17c and supplies it to the display unit 6. The display unit 6 can then display the fluorescence image information beside the fluorescence image information supplied from the first frame memory 17a.

Alternatively, the image-processing circuit 18 can extract the difference between the fluorescence image information received from the first frame memory 17a and that received from the third frame memory 17c and supply the difference information (image information subjected to correction for the effect of autofluorescence) to the red channel of the display unit 6. Thus, the third frame memory 17c and the image-processing circuit 18 constitute a correcting part for correcting for the effect of autofluorescence on the observation results of fluorescence emitted from the fluorescent dye deposited on or absorbed in the object A under examination, based on the correction information acquired by the correction-information acquiring part.

The feed unit 20 includes a first tank 21 storing cleaning water for cleaning an affected area, a second tank 22 storing a fluorescent dye/probe solution, a valve 23 for selectively supplying/stopping the liquids from the tanks 21 and 22, and a feed tube 24 connected to the valve 23 to supply the liquids to the front end 2a along the insertion part 2. The valve 23 is controlled by the valve control circuit 25 included in the control unit 5. The valve 23 is composed of, for example, a three-way valve. A front end 24a of the feed tube 24 is disposed at the front end 2a of the insertion part 2 so that it can apply the cleaning water or fluorescent dye/probe solution to the object A under examination. The feed tube 24 can also be a forceps channel provided in the insertion part 2.

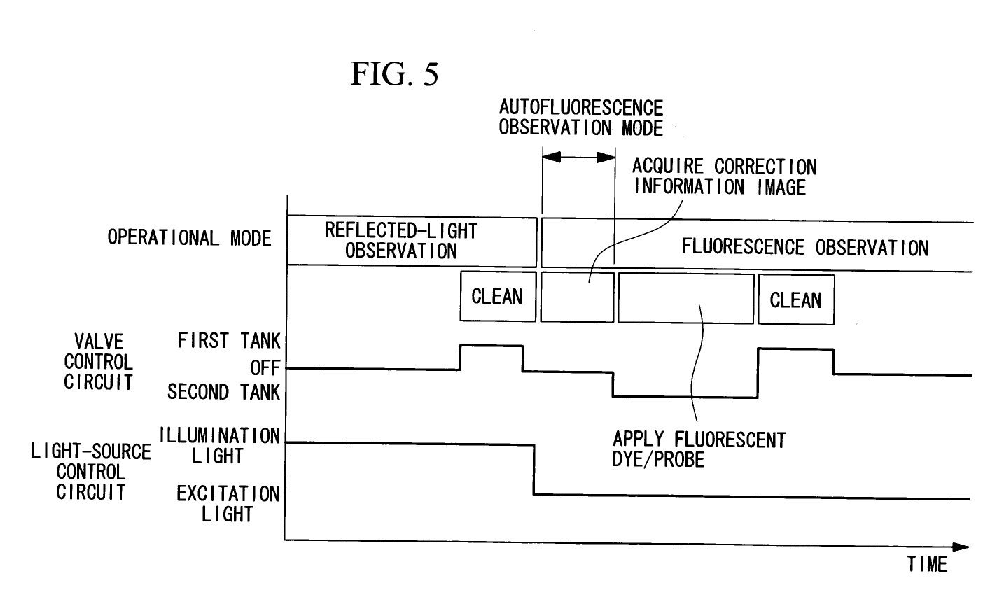

The valve control circuit 25 is connected to the light-source control circuit 10 so that at least the fluorescent dye/probe solution stored in the second tank 22 can be applied in synchronization with irradiation with excitation light from the excitation light source 9, as shown in FIG. 5.

The operation of the fluorescence endoscope system 1 according to this embodiment will now be described.

To acquire an image of the object A under examination in the body cavity using the fluorescence endoscope system 1 according to this embodiment, the insertion part 2 is inserted into the body cavity such that the front end 2a thereof faces the object A under examination (for example, a disease-suspected area of biological tissue in the body cavity). In this state, the light source unit 4 and the control unit 5 are operated such that the light-source control circuit 10 alternately drives the illumination light source 8 and the excitation light source 9 to emit illumination light and excitation light, respectively.

Before the image acquisition of the object A under examination, a fluorescent drug is applied thereto at an appropriate time to allow fluoroscopy. In this embodiment, the fluorescent drug is applied to the object A under examination between completion of the autofluorescence observation mode and observation of drug fluorescence, as described later.

In the image acquisition of the object A under examination, a mark such as a slight scratch may be formed on the object A under examination using, for example, biopsy forceps so as not to lose the position of the object A under examination.

The excitation light or illumination light emitted from the light source unit 4 propagates through the light guide 7 to the front end 2a of the insertion part 2 and exits the front end 2a to be incident on the object A under examination.

When the object A under examination is irradiated with the excitation light after the fluorescent drug is applied thereto, the light excites the fluorescent drug penetrating the object A under examination, so that it emits fluorescence. The fluorescence emitted from the object A under examination is collected by the image-acquisition optical system 11 of the image-acquisition unit 3 and is transmitted through the excitation-light cutting filter 12 to be incident on the tunable-spectrum device 13.

The tunable-spectrum device 13 can transmit the incident fluorescence because the tunable-spectrum-device control circuit 16 has switched the tunable-spectrum device 13 to the first mode in synchronization with the operation of the excitation light source 9 to sufficiently increase the transmittance in a wavelength range including that of the fluorescence. In this case, the object A under examination reflects part of the excitation light incident thereon, which then enters the image-acquisition unit 3 together with the fluorescence, although the excitation-light cutting filter 12 of the image-acquisition unit 3 blocks the excitation light and thus prevents it from entering the image-acquisition device 14.

The fluorescence passing through the tunable-spectrum device 13 enters the image-acquisition device 14, which thereby acquires fluorescence image information. The fluorescence image information thus acquired is stored in the first frame memory 17a and is supplied to the red channel of the display unit 6 by the image-processing circuit 18, so that the display unit 6 displays the information.

When the object A under examination is irradiated with the illumination light, the light is reflected at the surface of the object A under examination, is collected by the image-acquisition optical system 11, and is transmitted through the excitation-light cutting filter 12 to be incident on the tunable-spectrum device 13. Because the wavelength range of the reflected light of the illumination light lies in the fixed transmission range of the tunable-spectrum device 13, the tunable-spectrum device 13 transmits all of the reflected light incident thereon.

The reflected light passing through the tunable-spectrum device 13 enters the image-acquisition device 14, which thereby acquires reflected-light image information. The reflected-light image information thus acquired is stored in the second frame memory 17b and is supplied to the green channel of the display unit 6 by the image-processing circuit 18, so that the display unit 6 displays the information.

In this case, even if fluorescence enters the tunable-spectrum device 13, it blocks the fluorescence because the tunable-spectrum-device control circuit 16 has switched the tunable-spectrum device 13 to the second mode in synchronization with the operation of the illumination light source 8 to decrease the transmittance in the wavelength range of the fluorescence. The tunable-spectrum device 13 thus allows the image-acquisition device 14 to acquire an image of the reflected light alone.

In the fluorescence endoscope system 1 according to this embodiment, the light-source control circuit 10 and the valve control circuit 25 are operated so as to perform reflected-light observation prior to fluorescence observation. In the reflected-light observation, the light-source control circuit 10 drives the illumination light source 8 to irradiate the object A under examination with illumination light.

In switching from the reflected-light observation to the fluorescence observation, the valve control circuit 25 switches the valve 23 to the first tank 21 side while the illumination light source 8 is radiating illumination light before radiating excitation light. The cleaning water stored in the first tank 21 is then discharged from the front end 24a of the feed tube 24 toward the object A under examination, thus cleaning the surface thereof.

In this case, according to this embodiment, the object A under examination is cleaned while being irradiated with illumination light from the illumination light source 8. This facilitates checking of an affected area and allows cleaning of the area where the fluorescent dye is to be applied while being monitored.

Thus, endoscopy using the fluorescence endoscope system 1 allows quick determination of whether a suspected area is cancerous or not by reliably applying an esterase-sensitive fluorescent probe to the area. In this case, the esterase-sensitive fluorescent probe does not spread through the whole body via the blood, and a tumor can thus be quickly identified and examined at an appropriate time with a small amount of fluorescent probe. In contrast to oral administration or administration by intravenous injection (administration of a large amount of drug), for example, the amount of expensive fluorescent drug used can be minimized to reduce the cost of examination.

If the fluorescence observation performed after the reflected-light observation is the first fluorescence observation performed on the object A under examination, the operational mode of the fluorescence endoscope system 1 is shifted to the autofluorescence observation mode when switching from the reflected-light observation to the fluorescence observation to perform observation for correction-information acquisition (observation step used for correction-information acquisition).

In the autofluorescence observation mode, the valve control circuit 25 switches the valve 23 to the off position while the light-source control circuit 10 drives the excitation light source 9 to irradiate the object A under examination with excitation light.

As described above, the fluorescent drug is applied to the object A under examination after the completion of the autofluorescence observation mode. That is, the fluorescent drug has not yet been applied to the object A under examination in the autofluorescence observation mode, where the object A under examination is irradiated with excitation light to emit autofluorescence.

The wavelength of the excitation light used for irradiation of the object A under examination in the autofluorescence observation mode lies in the absorption spectrum of the fluorescent dye. In the autofluorescence observation mode, therefore, the object A under examination emits autofluorescence similar to the autofluorescence emitted in the fluorescence observation using the fluorescent dye.

The autofluorescence emitted from the object A under examination is collected by the image-acquisition optical system 11 of the image-acquisition unit 3 and is transmitted through the excitation-light cutting filter 12 to be incident on the tunable-spectrum device 13.

The tunable-spectrum device 13 can transmit the incident fluorescence in the autofluorescence observation mode because the tunable-spectrum-device control circuit 16 has switched the tunable-spectrum device 13 to the first mode in synchronization with the operation of the excitation light source 9 to sufficiently increase the transmittance in a wavelength range including that of the fluorescence. On the other hand, the excitation-light cutting filter 12 of the image-acquisition unit 3 blocks the excitation light and thus prevents it from entering the image-acquisition device 14.

The fluorescence passing through the tunable-spectrum device 13 enters the image-acquisition device 14, which thereby acquires autofluorescence image information (image serving as correction information) (correction-information acquiring step). The autofluorescence image information thus acquired is stored in the third frame memory 17c.

The completion of the autofluorescence observation mode is followed by the application of the fluorescent drug (application of the fluorescent dye/probe) to the object A under examination. Specifically, the valve control circuit 25 detects the completion of the autofluorescence observation mode to switch the valve 23 to the second tank 22 side. The fluorescent drug stored in the second tank 22 is then discharged from the front end 24a of the feed tube 24 toward the object A under examination.

In this case, according to this embodiment, a small amount of fluorescent dye can be accurately applied to the target area to be subjected to the fluorescence observation because the target area has been identified in the reflected-light observation prior to the fluorescence observation. In addition, even a transparent fluorescent dye can be reliably locally applied by monitoring the application conditions while the object A under examination is being irradiated with excitation light from the excitation light source 9.

Thus, the fluorescent drug is applied to the object A under examination to observe the fluorescence emitted from the fluorescent dye deposited on or absorbed in the object A under examination. The fluorescence observation of the object A under examination using the fluorescent drug is started after the lapse of a predetermined reaction time of the fluorescent drug until the fluorescence observation is enabled.

The image-processing circuit 18 receives the image information acquired by irradiation with excitation light in the autofluorescence observation from the third frame memory 17c, and the display unit 6 displays it beside the fluorescence image information supplied from the first frame memory 17a.

That is, the display unit 6 displays the fluorescence image information about the object A under examination before the application of the fluorescent drug (autofluorescence image information) beside the fluorescence image information on the object A under examination after the application of the fluorescent drug.

A comparison of the fluorescence image information allows discrimination between a region of the object A under examination where the fluorescence intensity has increased after the application of the fluorescent drug (region showing drug fluorescence) and a region where the fluorescence intensity has not changed after the application of the fluorescent drug (region showing no drug fluorescence).

The region showing drug fluorescence, where the fluorescent drug has reacted, can be determined to be an affected area.

The image-processing circuit 18 can extract the difference between the fluorescence image information received from the first frame memory 17a and that received from the third frame memory 17c (difference in fluorescence intensity) and supply the difference information to the red channel of the display unit 6. This difference information is fluorescence image information about only the fluorescence emitted from the fluorescent dye deposited on or absorbed in the object A under examination (fluorescence image information unaffected by autofluorescence). That is, the display unit 6 displays image information subjected to correction for the effect of autofluorescence on the observation results of the fluorescence emitted from the fluorescent dye deposited on or absorbed in the object A under examination (correction step).

Thus, the fluorescence endoscope system 1 according to this embodiment allows correction for the effect of autofluorescence on the results of the fluorescence observation using the fluorescent dye, based on the autofluorescence information (correction information) acquired in the autofluorescence observation mode. In this embodiment, therefore, superior fluoroscopy can be performed on the object A under examination while suppressing the effect of autofluorescence.

In addition, because the fluorescence endoscope system 1 according to this embodiment allows superior fluoroscopy, it contributes to a reduction in the amount of fluorescent drug required to ensure a sufficient S/N ratio between autofluorescence and fluorescence due to the fluorescent dye in the fluoroscopy. According to this embodiment, therefore, the amount of expensive fluorescent drug used can be reduced.

In addition, the fluorescence endoscope system 1 according to this embodiment acquires the correction information used to correct for the effect of autofluorescence on the results of fluorescence observation immediately before the observation of drug fluorescence. According to this embodiment, therefore, the accuracy of the results of fluorescence observation can be increased.

In the fluorescence endoscope system 1 according to this embodiment, additionally, the correction-information acquiring part used for acquiring the correction information also functions as the fluorescence-information acquiring part used for the observation of drug fluorescence (image-acquisition unit 3). According to this embodiment, therefore, the number of components of the fluorescence endoscope system 1 can be reduced to lower manufacturing costs.

In addition, the fluorescence endoscope system 1 according to this embodiment can provide the user with a combined image of fluorescence and reflected-light images.

The fluorescence endoscope system 1 according to this embodiment includes the tunable-spectrum device 13. The transmittance characteristics of the tunable-spectrum device 13 can be changed merely by changing the distance between the planar optical members 13a and 13b. Therefore, the tunable-spectrum device 13 and the image-acquisition device 14, which are extremely small, can be accommodated at the front end 2a of the insertion part 2. According to this embodiment, therefore, the fluorescence or reflected light from the object A under examination does not have to be guided out of the body through, for example, a fiber bundle.

In addition to weak fluorescence images, which tend to be degraded in quality due to, for example, noise, the fluorescence endoscope system 1 according to this embodiment can acquire other images to allow efficient examination of an affected area.

In this embodiment, the mode of the tunable-spectrum device 13 is switched in synchronization with the switching between the light sources 8 and 9 of the light source unit 4, so that images of light in different wavelength ranges can be acquired using the same image-acquisition device 14. According to this embodiment, therefore, a plurality of image-acquisition optical systems corresponding to fluorescence or reflected light does not have to be used, and thus the diameter of the insertion part 2 can be reduced.

Noise reduction is particularly important in observing weak light such as fluorescence because external light capable of passing through biological tissue is present even in a body cavity of a living organism. In this embodiment, the tunable-spectrum device 13 of the image-acquisition unit 3 can always block light of wavelengths other than that of the fluorescence from the object A under examination irrespective of the wavelength range under observation, thus acquiring a superior image with reduced noise.

In this embodiment, the illumination light source 8 emits illumination light in the wavelength range of 420 to 450 nm, which includes the absorption wavelength range of hemoglobin. Accordingly, the reflected light can be subjected to image acquisition to acquire information about, for example, the structure of blood vessels present relatively close to the surface of the body.

In general, light of longer wavelengths is less susceptible to the effects of scattering inside a living organism. Such light can be relatively easily observed even if the light is fluorescence emitted from a portion deep inside the body. Light with a wavelength of 1 μm or more, however, is difficult to observe because such light is attenuated by absorption in moisture. The fluorescence endoscope system 1 according to this embodiment can use a fluorescent dye that emits fluorescence in the near-infrared region to efficiently acquire in vivo information, particularly, information about a disease, such as cancer, occurring near the mucous membrane.

In the image-acquisition unit 3 of the fluorescence endoscope system 1 according to this embodiment, the image-acquisition optical system 11, the excitation-light cutting filter 12, and the tunable-spectrum device 13 are arranged in the above order from near the front end 2a side of the insertion portion 2, although the order of arrangement of these components is not limited to the above order, and any order can be used.

The fluorescence endoscope system 1 according to this embodiment uses a fluorescein-based esterase-sensitive fluorescent probe, although a cyanine-based compound, such as a tricarbocyanine-based fluorescent probe, can be used in combination with or instead of the esterase-sensitive fluorescent probe. Such diagnostic drugs or contrast media can be used in the present invention.

In image acquisition inside a body cavity of a living organism, generally, the luminance of a drug fluorescence image is much lower than that of a reflected-light image. This can result in the need to appropriately adjust the amount of light (exposure level) entering the image-acquisition device 14 each time the reflected-light image or the drug fluorescence image is acquired.

In addition to the switching of the irradiation light (excitation light) of the light source unit 4 and the spectral properties of the tunable-spectrum device 13, the control unit 5 preferably adjusts the exposure level of the image-acquisition unit 3 (image-acquisition device 14) during the image acquisition according to the image luminance measured by the image-acquisition device 14 so that the image luminance approaches a predetermined target level. To adjust the exposure level, specifically, the control unit 5 preferably performs at least one of adjustment of illumination light (excitation light) from the light source unit 4 (adjustment of emission intensity or duration), adjustment of the exposure of the image-acquisition unit 3 (adjustment of shutter speed or aperture), and adjustment of the gain of the image-acquisition unit 3.

Such adjustments have greater importance when a single image is created from a plurality of images with widely differing luminances and high-luminance regions (bright regions), for example, a combination of a reflected-light image with relatively high luminance over the entire image and a drug fluorescence image whose fluorescence region is limited (administered) to a region where the drug is applied.

In the above image luminance adjustment, the image luminance can be measured either in an average photometry mode where the average luminance of an entire image or part thereof is determined to be the luminance of the image or in a peak photometry mode where the maximum luminance of an entire image or part thereof is determined to be the luminance of the image.

Preferably, the autofluorescence image (correction information image) is acquired in the peak photometry mode so that no halation occurs in the entire image.

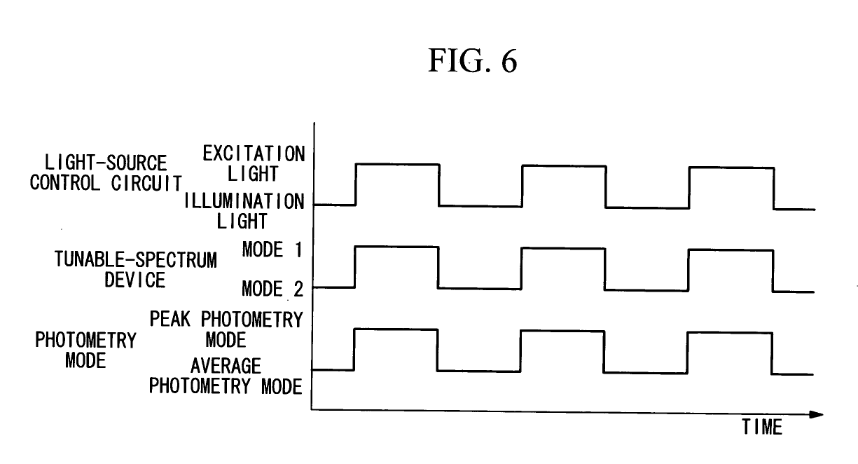

More preferably, the photometry mode is controlled in association with the operation of the light-source control circuit 10 and the tunable-spectrum-device control circuit 16 at a predetermined timing according to a timing chart shown in FIG. 6 such that the photometry mode is switched to the average photometry mode on acquiring an reflected-light image and is switched to the peak photometry mode on acquiring a drug fluorescence image.

The average photometry mode is effective in acquiring a reflected-light image because a subject often occupies most of the image to form a relatively bright region over the entire image. If the reflected-light image is acquired by peak photometry, the object A under examination would be darkened because the luminance would be adjusted so that the luminance of an extremely bright region due to, for example, light reflected from bodily fluid approaches the target level.

The peak photometry mode, on the other hand, is effective in acquiring a drug fluorescence image because its fluorescent region is limited to the region where the drug is applied (administered). In this case, a dark region without fluorescence often occupies most of the image, and drug fluorescence is observed only in part of the image.

If average photometry is employed, a hardly observable image with emphasized noise in the region without fluorescence would result because the luminance would be adjusted so that the average luminance including the luminance of the dark region occupying most of the image approaches the target level.

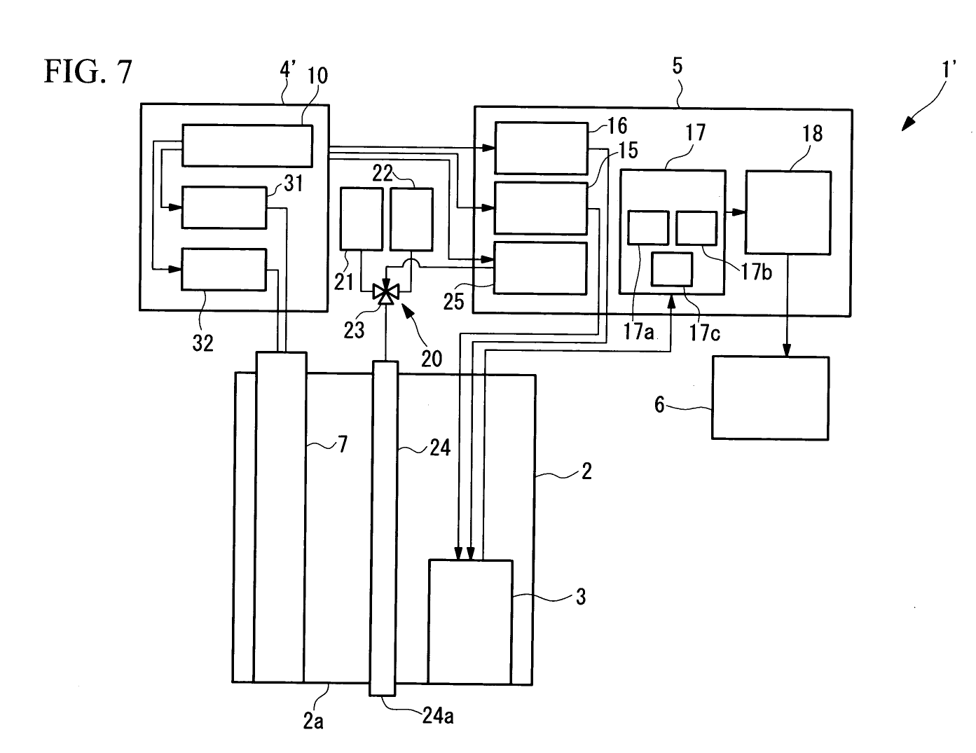

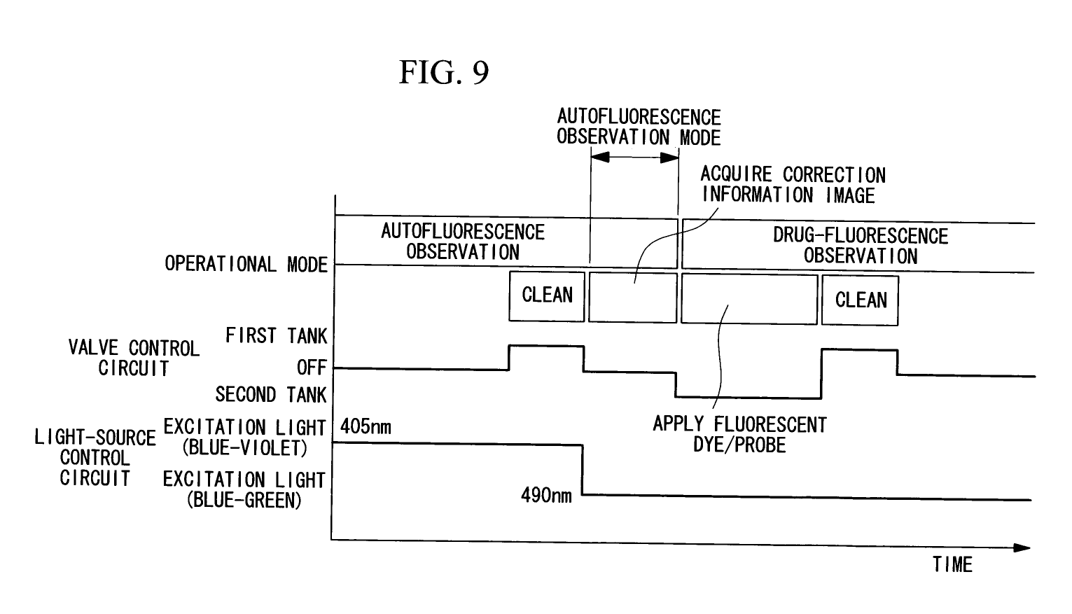

Next, a fluorescence endoscope system 1′ according to a second embodiment of the present invention will be described with reference to FIGS. 7 to 9.

In the description of the second embodiment, the same parts as used in the above fluorescence endoscope system 1 according to the first embodiment are indicated by the same reference numerals, and no description thereof will be given.

The fluorescence endoscope system 1′ according to this embodiment differs from the fluorescence endoscope system 1 according to the first embodiment in the configuration of a light source unit 4′ and the transmittance characteristics of the tunable-spectrum device 13 and the excitation-light cutting filter 12.

Referring to FIG. 7, the light source unit 4′ of the fluorescence endoscope system 1′ according to this embodiment includes two excitation light sources 31 and 32.

The first excitation light source 31 is a semiconductor laser that emits first excitation light with a peak wavelength of 490±5 nm. The first excitation light can excite a fluorescein-based esterase-sensitive fluorescent probe.

The second excitation light source 32 is a semiconductor laser that emits second excitation light with a peak wavelength of 405±5 nm. The second excitation light can excite biological autofluorescence of, for example, collagen, NADH, or FAD.

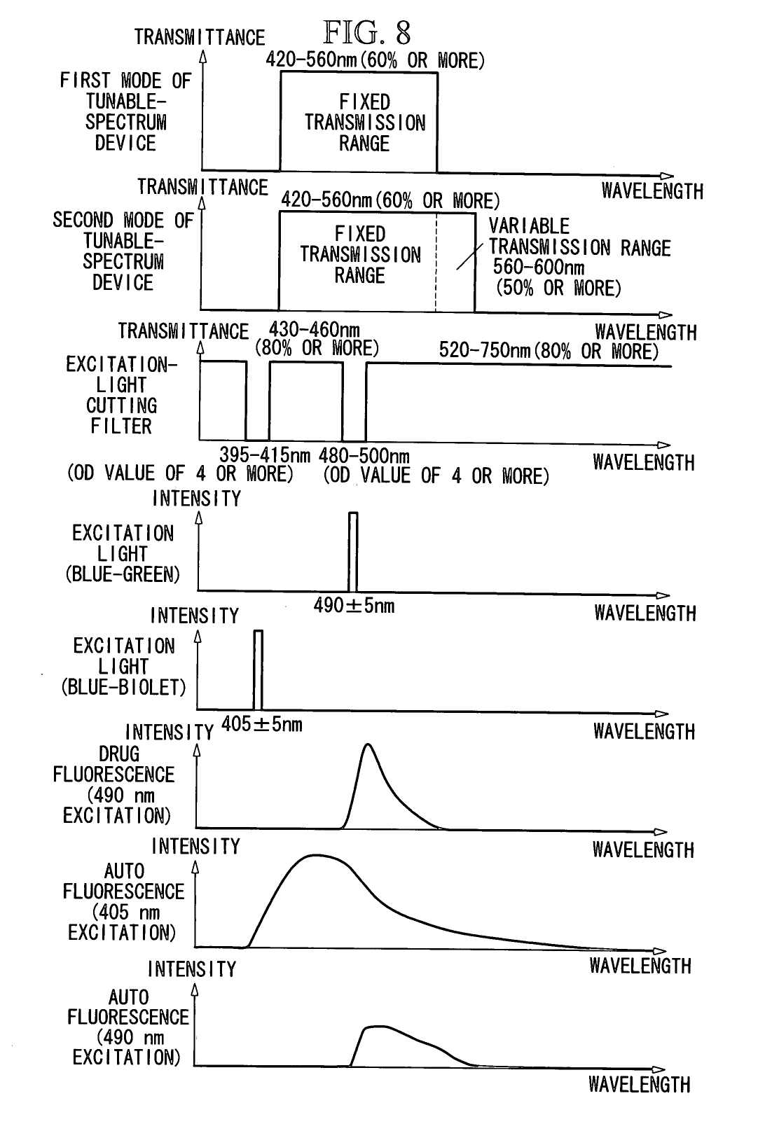

Referring to FIG. 8, the tunable-spectrum device 13 has drug fluorescence and a variable transmission range which can be switched between two modes: a first mode where the transmittance is high in a fixed transmission range including the drug fluorescence and the short-wavelength range of the autofluorescence and where the transmittance is low in the long-wavelength range of the autofluorescence; and a second mode where the transmittance is high in the long-wavelength range of the autofluorescence and in the fixed transmission range.

The first mode is a mode where the tunable-spectrum device 13 transmits drug fluorescence. In the first mode, the transmittance in the variable transmission range is sufficiently decreased in comparison with the second mode to block autofluorescence in the variable transmission range, which would cause noise in drug fluorescence observation.

The fixed transmission range is set to the range of, for example, 420 to 560 nm, with a transmittance of 60% or more. The variable transmission range is set to the range of 560 to 600 nm, with a transmittance of 50% or more in the second mode. In the first mode, the variable transmission range is shifted into the fixed transmission range. The variable transmission range can also be set to a wavelength range including the peak wavelength of porphyrin, an autofluorescent component (for example, 620 to 660 nm).

The excitation-light cutting filter 12 has an OD of 4 or more (=a transmittance of 1×10-4 or less) in the wavelength range of 395 to 415 nm, a transmittance of 80% or more in the wavelength range of 430 to 460 nm, an OD of 4 or more (=a transmittance of 1×10-4 or less) in the wavelength range of 480 to 500 nm, and a transmittance of 80% or more in the wavelength range of 520 to 750 nm.

In the fluorescence endoscope system 1′ according to this embodiment, when the light-source control circuit 10 drives the first excitation light source 31 to emit the first excitation light, the operation of the second excitation light source 32 is stopped, and the object A under examination is irradiated only with the first excitation light. The tunable-spectrum-device control circuit 16 then switches the tunable-spectrum device 13 to the first mode in synchronization with the operation of the first excitation light source 31. Accordingly, drug fluorescence emitted from the object A under examination passes through the tunable-spectrum device 13, so that the image-acquisition device 14 acquires an image of the drug fluorescence. Thus, drug fluorescence image information is stored in the first frame memory 17a.

On the other hand, when the light-source control circuit 10 drives the second excitation light source 32 to emit the second excitation lights the operation of the first excitation light source 31 is stopped, and the object A under examination is irradiated only with the second excitation light. The tunable-spectrum-device control circuit 16 then switches the tunable-spectrum device 13 to the second mode in synchronization with the operation of the second excitation light source 32. Accordingly, autofluorescence (excitation wavelength: 405 nm) emitted from the object A under examination passes through the tunable-spectrum device 13, so that the image-acquisition device 14 acquires an image of the autofluorescence. Thus, autofluorescence image information is stored in the second frame memory 17b.

The drug fluorescence image information stored in the first frame memory 17a is supplied to, for example, the red channel of the display unit 6 by the image-processing circuit 18, so that the display unit 6 displays the information.

On the other hand, the autofluorescence image information stored in the second frame memory 17b is supplied to, for example, the green channel of the display unit 6 by the image-processing circuit 18, so that the display unit 6 displays the information. The user can thus be provided with an image in which the fluorescence image and the autofluorescence image (excitation wavelength: 405 nm) are combined, and therefore, the fluorescence endoscope system 1′ that acquires a plurality of images carrying different information can be provided.

In the fluorescence endoscope system 1′ according to this embodiment, the light-source control circuit 10 and the valve control circuit 25 are operated so as to perform autofluorescence observation (excitation wavelength: 405 nm) prior to drug fluorescence observation. In the autofluorescence observation, the light-source control circuit 10 drives the second excitation light source 32 to irradiate the object A under examination with the second excitation light.

In switching from the autofluorescence observation to the drug fluorescence observation, the valve control circuit 25 switches the valve 23 to the first tank 21 side while the second excitation light source 32 is radiating the second excitation light being radiating the first excitation light. The cleaning water stored in the first tank 21 is then discharged from the front end 24a of the feed tube 24 toward the object A under examination, thus cleaning the surface thereof.

In this case, according to this embodiment, the object A under examination is cleaned while being irradiated with the second excitation light from the second excitation light source 32. Therefore, the autofluorescence facilitates checking of an affected area and allows cleaning of the area where the fluorescent dye is to be applied while being monitored.

Subsequently, when the light-source control circuit 10 drives the first excitation light source 31 to irradiate the object A under examination with the first excitation light, the valve control circuit 25 switches the valve 23 to the second tank 22 side in response to a signal transmitted from the light-source control circuit 10. The fluorescent drug stored in the second tank 22 is then discharged from the front end 24a of the feed tube 24 toward the object A under examination.

In this case, according to this embodiment, a small amount of fluorescent dye can be accurately applied to the target area to be subjected to the fluorescence observation because the target area has been identified in the autofluorescence observation prior to the fluorescence observation. In addition, the fluorescent dye is applied while the object A under examination is being irradiated with the first excitation light from the first excitation light source 31. According to this embodiment, therefore, even a transparent fluorescent dye can be reliably locally applied while monitoring the application conditions.

If the drug fluorescence observation performed after the autofluorescence observation is the first drug fluorescence observation performed on the object A under examination, the operational mode of the fluorescence endoscope system 1′ is switched to the autofluorescence observation mode when switching from the autofluorescence observation to the drug fluorescence observation to perform observation for correction-information acquisition (observation step used for correction-information acquisition).

In the autofluorescence observation mode, the valve control circuit 25 switches the valve 23 to the off position while the light-source control circuit 10 drives the first excitation light source 31 to irradiate the object A under examination with the first excitation light.

As described above, the fluorescent drug is applied to the object A under examination after the completion of the autofluorescence observation mode. That is, the fluorescent drug has not yet been applied to the object A under examination in the autofluorescence observation mode, where the object A under examination is irradiated with the first excitation light to emit autofluorescence (excitation wavelength: 490 nm).

The autofluorescence (excitation wavelength: 490 nm) emitted from the object A under examination is collected by the image-acquisition optical system 11 of the image-acquisition unit 3 and is transmitted through the excitation-light cutting filter 12 to be incident on the tunable-spectrum device 13.

In the autofluorescence observation mode, the tunable-spectrum-device control circuit 16 has switched the tunable-spectrum device 13 to the first mode in synchronization with the operation of the first excitation light source 31. Accordingly, the autofluorescence (excitation wavelength: 490 nm) passes through the tunable-spectrum device 13 to be incident on the image-acquisition device 14, which thereby acquires autofluorescence image information (image serving as correction information; excitation wavelength: 490 nm) (correction-information acquiring step). The autofluorescence image information (excitation wavelength: 490 nm) is stored in the third frame memory 17c.

The completion of the autofluorescence observation mode is followed by the application of the fluorescent drug (application of the fluorescent dye/probe) to the object A under examination.

Subsequently, the image-processing circuit 18 receives the fluorescence image information acquired by irradiation with the first excitation light in the autofluorescence observation (excitation wavelength: 490 nm) from the third frame memory 17c, and the display unit 6 displays it beside the fluorescence image information supplied from the first frame memory 17a.

Alternatively, the image-processing circuit 18 extracts the difference between the fluorescence image information received from the first frame memory 17a and that received from the third frame memory 17c (difference in fluorescence intensity) and supplies the difference information to the red channel of the display unit 6. That is, the display unit 6 displays image information subjected to correction for the effect of autofluorescence on the observation results of the fluorescence emitted from the fluorescent dye deposited on or absorbed in the object A under examination (correction step).

Thus, the fluorescence endoscope system 1′ according to this embodiment allows correction for the effect of autofluorescence on the results of fluorescence observation using the fluorescent dye, based on the autofluorescence information (correction information) acquired in the autofluorescence observation mode. In this embodiment, therefore, superior fluoroscopy can be performed on the object A under examination while suppressing the effect of autofluorescence.

In addition, because the fluorescence endoscope system 1′ according to this embodiment allows superior fluoroscopy, it contributes to a reduction in the amount of fluorescent drug required to ensure a sufficient S/N ratio between autofluorescence and fluorescence due to the fluorescent dye in the fluoroscopy. According to this embodiment, therefore, the amount of expensive fluorescent drug used can be reduced.

The fluorescence endoscope system 1′ according to this embodiment uses a fluorescein-based esterase-sensitive fluorescent probe, although a cyanine-based fluorescent dye/probe, such as a tricarbocyanine-based fluorescent probe, can be used instead of the esterase-sensitive fluorescent probe.

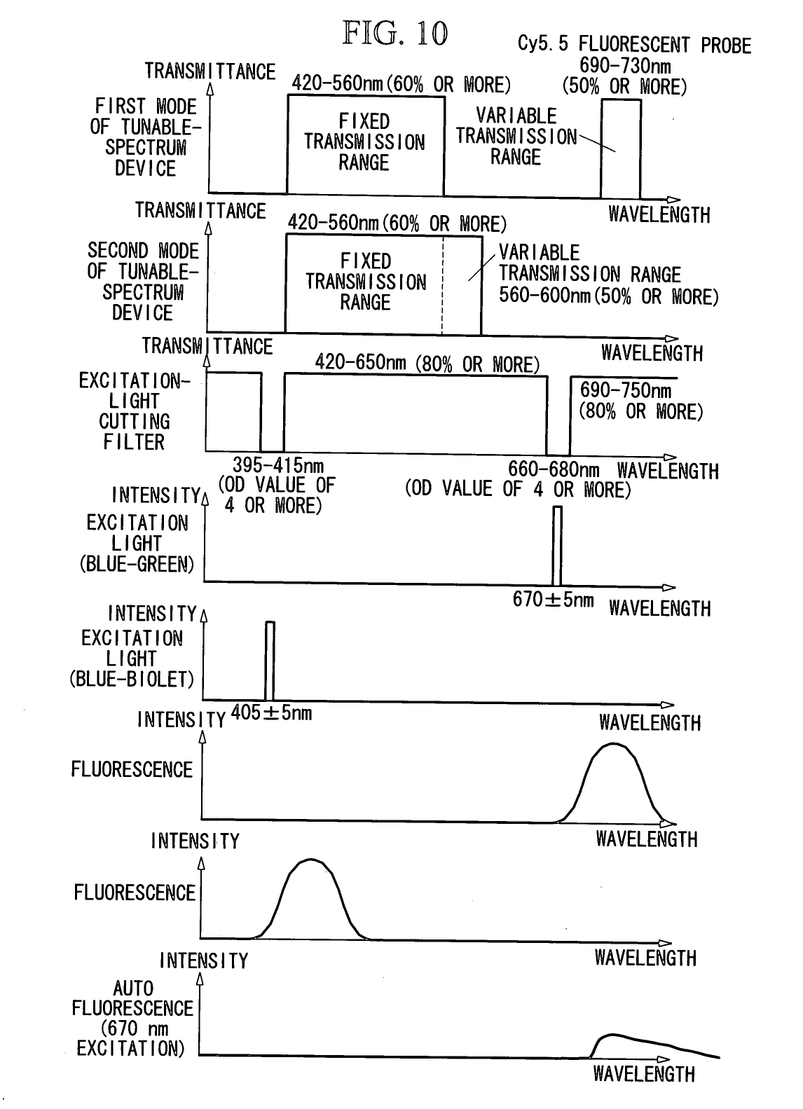

In this case, as shown in FIG. 10, the variable transmission range of the tunable-spectrum device 13 can be shifted to a wavelength range including the wavelength of fluorescence (drug fluorescence) emitted from the fluorescent dye/probe through excitation by excitation light (for example, 690 to 730 nm). In the first mode, the transmittance in the variable transmission range is increased to 50% or more so that the tunable-spectrum device 13 transmits the drug fluorescence. In the second mode, the variable transmission range is shifted to, for example, 560 to 600 nm so that the tunable-spectrum device 13 blocks the drug fluorescence while transmitting the autofluorescence.

In this case, the excitation-light cutting filter 12 has an OD of 4 or more (=a transmittance of 1×10-4 or less) in the wavelength range of 395 to 415 nm, a transmittance of 80% or more in the wavelength range of 420 to 650 nm, an OD of 4 or more (=a transmittance of 1×10-4 or less) in the wavelength range of 660 to 680 nm, and a transmittance of 80% or more in the wavelength range of 690 to 750 nm.

The first excitation light source 31 is, for example, a semiconductor laser that emits excitation light with a peak wavelength of 670±5 nm. With this wavelength, the excitation light can excite a cyanine-based fluorescent dye/probe such as a tricarbocyanine-based fluorescent probe.

Thus, the fluorescence endoscope system 1′ according to this embodiment can achieve the same advantages as in the case where a fluorescein-based esterase-sensitive fluorescent probe is used.

The type of the fluorescence endoscope systems 1 and 1′ according to the present invention is not limited to an endoscope including an insertion part 2, for insertion into a body cavity of a living organism, having an image-acquisition device 14 at a front end thereof, but can also be applied to a capsule endoscope including a casing that accommodates a light source part, an image-acquisition part, and a tunable-spectrum part and that can be inserted into a body cavity of a living organism.

In addition, the present invention is not limited to a fluorescence endoscope system that is partially inserted into a body cavity, but can also be applied to, for example, a fluoroscopy apparatus for fluoroscopy of an exposed object under examination, a fluoroscopy apparatus for fluoroscopy of an object under examination extracted and removed from a living organism, or a fluorescence-information processing apparatus for processing information about fluorescence emitted from a fluorescent dye deposited on or absorbed in an object under examination.

For example, the present invention can at least be applied to a fluoroscopy apparatus including an excitation-light radiating part configured to irradiate an object under examination with excitation light having a wavelength included in an absorption spectrum of a fluorescent dye in a state where the fluorescent dye is not deposited on or absorbed in the biological tissue; and a correction-information acquiring part configured to acquire correction information that can be used to correct for the effect of autofluorescence of the object under examination on observation results of fluorescence emitted from the fluorescent dye deposited on or absorbed in the object under examination upon irradiation with the excitation light, based on observation results of the object under examination upon irradiation with the excitation light by the excitation-light radiating part.

In addition, the present invention can at least be applied to a fluoroscopy apparatus including an excitation-light radiating part configured to irradiate an object under examination extracted and removed from a living organism with excitation light having a wavelength included in an absorption spectrum of a fluorescent dye deposited on or absorbed in the object under examination in a state where the fluorescent dye is not deposited on or absorbed in the object under examination; and a correction-information acquiring part configured to acquire correction information that can be used to correct for the effect of autofluorescence of the object under examination on observation results of fluorescence emitted from the fluorescent dye deposited on or absorbed in the object under examination upon irradiation with the excitation light, based on observation results of the object under examination upon irradiation with the excitation light by the excitation-light radiating part.

In addition, the present invention can at least be applied to a fluorescence-information processing apparatus including a correction-information storing part configured to store information about observation results of an object under examination upon irradiation with excitation light having a wavelength included in an absorption spectrum of a fluorescent dye in a state where the fluorescent dye is not deposited on or absorbed in the object under examination; and a correcting part configured to correct for the effect of autofluorescence of the object under examination on observation results of fluorescence emitted from the fluorescent dye deposited on or absorbed in the biological tissue upon irradiation with the excitation light, based on the information stored in the correction-information storing part.

What is claimed is:

1. A fluorescence endoscope system for observing fluorescence emitted from a fluorescent dye deposited on or absorbed in biological tissue in a body cavity of a living organism by inserting at least part of the system into the body cavity and irradiating the biological tissue with excitation light, the system comprising:
an excitation-light radiating part configured to irradiate the biological tissue with excitation light having a wavelength included in an absorption spectrum of the fluorescent dye in a state where the fluorescent dye is not deposited on or absorbed in the biological tissue; and
a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

2. The fluorescence endoscope system according to claim 1, wherein the state where the fluorescent dye is not deposited on or absorbed in the biological tissue is a state before the fluorescent dye is deposited on or absorbed in the biological tissue.

3. The fluorescence endoscope system according to claim 1, wherein the correction-information acquiring part also functions as a fluorescence-information acquiring part configured to acquire information about the fluorescence emitted from the fluorescent dye deposited on or absorbed in the biological tissue.

4. The fluorescence endoscope system according to claim 1, wherein the correction-information acquiring part also functions as an image-acquisition part configured to acquire information about the fluorescence emitted from the fluorescent dye deposited on or absorbed in the biological tissue as image information.

5. The fluorescence endoscope system according to claim 1, further comprising a correction-information storing part configured to store the correction information acquired by the correction-information acquiring part.

6. The fluorescence endoscope system according to claim 1, further comprising a correcting part configured to correct for the effect of autofluorescence of the biological tissue on the observation results of the fluorescence emitted from the fluorescent dye deposited on or absorbed in the biological tissue, based on the correction information acquired by the correction-information acquiring part.

7. The fluorescence endoscope system according to claim 1, wherein the fluorescent dye has the ability to selectively stain normal cells and tumor cells in the biological tissue.

8. A fluoroscopy apparatus for observing fluorescence emitted from a fluorescent dye deposited on or absorbed in biological tissue in a body cavity of a living organism by irradiating the biological tissue with excitation light, the apparatus comprising:
an excitation-light radiating part configured to irradiate the biological tissue with excitation light having a wavelength included in an absorption spectrum of the fluorescent dye in a state where the fluorescent dye is not deposited on or absorbed in the biological tissue; and
a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

9. A fluoroscopy apparatus for observing fluorescence emitted from a fluorescent dye deposited on or absorbed in biological tissue extracted and removed from a living organism by irradiating the biological tissue with excitation light, the apparatus comprising:
an excitation-light radiating part configured to irradiate the biological tissue with excitation light having a wavelength included in an absorption spectrum of the fluorescent dye in a state where the fluorescent dye is not deposited on or absorbed in the biological tissue; and
a correction-information acquiring part configured to acquire correction information that can be used to correct for the effect of autofluorescence of the biological tissue on observation results of the fluorescence emitted from the fluorescent dye deposited on or absorbed in the biological tissue upon irradiation with the excitation light, based on observation results of the biological tissue upon irradiation with the excitation light by the excitation-light radiating part.

10. A fluoroscopy method for observing fluorescence emitted from a fluorescent dye deposited on or absorbed in biological tissue extracted and removed from a living organism by irradiating the biological tissue with excitation light, the method comprising:
an observation step, used for correction-information acquisition, in which the biological tissue is irradiated with excitation light having a wavelength included in an absorption spectrum of the fluorescent dye in a state where the fluorescent dye is not deposited on or absorbed in the biological tissue and observation results of the biological tissue upon irradiation with the excitation light are acquired; and
a correction-information acquiring step of acquiring correction information that can be used to correct for the effect of autofluorescence of the biological tissue on the observation results of the fluorescence emitted from the fluorescent dye deposited on or absorbed in the biological tissue upon irradiation with the excitation light, based on the observation results, acquired in the observation step used for correction-information acquisition, of the biological tissue upon irradiation with the excitation light.

11. A fluorescence-information processing apparatus for processing information about fluorescence emitted from a fluorescent dye deposited on or absorbed in biological tissue by irradiating the biological tissue with excitation light, the apparatus comprising:
a correction-information storing part configured to store information about observation results of the biological tissue upon irradiation with excitation light having a wavelength included in an absorption spectrum of the fluorescent dye in a state where the fluorescent dye is not deposited on or absorbed in the biological tissue; and
a correcting part configured to correct for the effect of autofluorescence of the biological tissue on observation results of the fluorescence emitted from the fluorescent dye deposited on or absorbed in the biological tissue upon irradiation with the excitation light, based on the information stored in the correction-information storing part.

12. A fluorescence-information processing method for processing information about fluorescence emitted from a fluorescent dye deposited on or absorbed in biological tissue by irradiating the biological tissue with excitation light, the method comprising:
a correction-information acquiring step of acquiring information about observation results of the biological tissue upon irradiation with excitation light having a wavelength included in an absorption spectrum of the fluorescent dye in a state where the fluorescent dye is not deposited on or absorbed in the biological tissue; and
a correction step of correcting for the effect of autofluorescence of the biological tissue on the observation results of the fluorescence emitted from the fluorescent dye deposited on or absorbed in the biological tissue upon irradiation with the excitation light, based on the information about the observation results acquired in the correction-information acquiring step.
